(19) United States
(12) Patent Application Publication
(10) Pub. No.: US 2016/0331834 A1
(43) Pub. Date: Nov. 17, 2016

(54) EPICUTANEOUS IMMUNOREBALANCING

(71) Applicant: DBV TECHNOLOGIES, MONTROUGE (FR)

(72) Inventors: LUCIE MONDOULET, CHATILLON (FR); VINCENT DIOSZEGHY, VILLIERS SUR MARNE (FR); PIERRE HENRI BENHAMOU, PARIS (FR); CHRISTOPHE DUPONT, CLAMART (FR)

(21) Appl. No.: 15/111,204

(22) PCT Filed: Jan. 14, 2015
(86) PCT No.: PCT/EP2015/050594

§ 371 Date: Jul. 13, 2016

Related U.S. Application Data
(60) Provisional application No. 61/928,716, filed on Jan. 17, 2014.

(51) Int. Cl. A61K 39/35; C12N 5/0783

(52) U.S. Cl. CPC A61K 39/35; C12N 5/0637; A61K 2039/54

(57) ABSTRACT

The present invention relates to compositions and methods for improving a subject condition by epicutaneous immunobalancing. The invention shows that particular regulatory T cells can be induced and maintained in a subject by epicutaneous treatment, thereby causing a substantial improvement in a subject condition. The invention may be used in a preventive context, to improve the immunobalance of a subject and avoid the onset or development of diseases, as well as in a therapeutic context, to improve a subject recovery. The invention is particularly suitable to prevent or treat a proliferative or autoimmune disease. The invention may be used in any mammalian subject, preferably in human subjects, including children and adults.

The present invention relates to compositions and methods for improving a subject condition by epicutaneous immunorebalancing. The invention shows that particular subsets of regulatory T cells can be induced and maintained in a subject by epicutaneous treatment, thereby causing a substantial improvement in a subject condition. The invention may be used in a preventive context, to improve the immunobalance of a subject and avoid the onset or development of diseases, as well as in a therapeutic context, to improve a subject recovery. The invention is particularly suitable to prevent or treat proliferative, autoimmune or inflammatory diseases, including allograft rejection. The invention may be used in any mammalian subject, preferably in human subjects, including children and adults.

### BACKGROUND OF THE INVENTION

Epicutaneous immunotherapy is a method of desensitizing an allergic subject by skin application of an allergen. By applying the allergen, in constant or varying doses, the subject becomes tolerant to the allergen. This method typically comprises the repeated application of an allergen known to cause an existing allergy on the skin of a subject, generally leading to diffusion of the allergen in the surface layers of the skin. Experiments conducted by the inventors have shown that the type of immune response generated by epicutaneous immunotherapy may be controlled by the treatment conditions. For example, commonly assigned international application WO2009/080934 discloses that a potent desensitization to an allergen may be obtained by epicutaneous therapy using an allergen, without adjuvant, applied on an intact area of the skin. Similarly, commonly assigned international application WO2009/071599 discloses that a strong desensitization to groundnut may be obtained by epicutaneous immunotherapy.

Although the mechanism of action of immunotherapy is not fully understood, it appears to involve several pathways such as (a) an increase in IgGs and, in particular, in IgG4 fractions, which are able to block the biological effects of IgEs; (b) a modification of the TH1 and TH2 immunological responses, promoting a more balanced TH1/TH2 response; and/or (c) a production of interleukin 10-producing T cells which display anti-allergic properties against mastocytes, certain T lymphocytes and eosinophils and also promote the production of IgG4.

Continuing their research, the applicant has found that some anti-allergic effects of epicutaneous immunotherapy could be mediated by the generation of regulatory T cells (“Tregs”). In particular, Applicant has discovered that certain Tregs could be induced by epicutaneous treatment with peanut allergen of a subject allergic to peanut, and that such an induction could contribute to the peanut desensitization (see J Immunol. 2011; 186(10):5629-37.). An epicutaneous method for preventing allergies has also been proposed in WO2013/117519, based on epicutaneous application of allergens.

Bynoe et al also described a method for preventing an allergic response in a subject by epicutaneous application of autoantigenic peptides (Immunity Vol 19 (2003), 317). According to this publication, such epicutaneous treatment yielded antigen-specific CD25⁻ suppressive T cells.

While the above publications relate to epicutaneous-mediated allergen-specific desensitization of allergic subjects, preliminary work was conducted to explore the ability to use epicutaneous immunotherapy for avoiding autoimmune disorders. In this regard, Strid et al (PLoS ONE 2(4) 2007 p e387) proposed an immunization with type II collagen but concluded that the levels of CD25+ T cells were not increased by the treatment.

Bohle B, et al. (The Journal of allergy and clinical immunology. 2007; 120(3):707-13), Francis J N, et al (Journal of allergy and clinical immunology. 2008; 121(5):1120-5) and Mobs C, et al (The e2. Epub 2008/04/01) suggest that sublingual or subcutaneous immunotherapies can induce IL-10-producing T regulatory cells (Tr1). The mechanism of action for Tr1 is only mediated by the secretion of IL-10. All authors have demonstrated that TGF-β or cell contact were not involved in the immune suppression effect. Furthermore, Bohle et al. described that IL-10-producing Tregs were induced during the early phase of SLIT but were not maintained during the later phase of SLIT. There is no disclosure or suggestion in the art of the possibility to treat existing non-allergic disorders by epicutaneous therapy, or to produce long-lasting and profound immunorebalancing in a subject.

### SUMMARY OF THE INVENTION

The present invention provides evidence that epicutaneous therapy can be used to treat or prevent deficits resulting from central or peripheral tolerance in subjects. The invention shows that particular regulatory T cell subsets having a remarkable phenotype, different from IL10+ Tregs observed during other immunotherapies, can be induced and maintained in a subject by epicutaneous treatment, thereby causing a substantial improvement in a subject condition. The invention may be used in a preventive context, to avoid the onset or development of diseases, as well as in a therapeutic context, to improve a subject recovery. The invention is particularly suitable to prevent or treat proliferative, autoimmune, allergic or inflammatory diseases.

It is therefore an object of the invention to provide a method for improving a subject condition, the method comprising continuously applying an antigen epicutaneously to an area of the skin of the subject. More preferably, the method comprises the continuous epicutaneous application of the antigen under conditions sufficient to induce (CTLA-4+, CD4+, CD25+) Foxp3+ Treg cells and/or to induce a modulation of methylation status of gene promoters, particularly an increase of CpG island methylation of GATA-3 gene. The method is particularly effective to prevent or treat a proliferative, autoimmune or inflammatory disease or any deficit resulting from central or peripheral tolerance. In this regard, the invention is also suitable to treat or prevent allergies by producing stable long-lasting tolerance mediated by Tregs and epigenetic modification in GATA-3 gene.

In this regard, a particular object of the invention relates to a method for preventing or reducing the onset or progression of a proliferative, autoimmune or inflammatory disease in a subject, comprising continuously applying an antigen epicutaneously to an area of the skin of the subject, said application leading to a prevention or reduction of said disease. The application is preferably performed under conditions sufficient to induce (CTLA-4+, CD4+, CD25+) Foxp3+ Treg cells and/or to modulate CpG island methylation of transcription factor DNA.

A further particular object of the invention relates to a method for treating or alleviating an existing proliferative, autoimmune or inflammatory disease in a subject, comprising continuously applying an antigen epicutaneously to an area of the skin of the subject, said application leading to a treatment or alleviation of said disease. The application is preferably performed under conditions sufficient to induce (CTLA-4+, CD4+, CD25+) Foxp3+ Treg cells and/or to modulate CpG island methylation of transcription factor DNA.

A further particular object of the invention relates to a method for preventing, treating or alleviating an allergy in a subject, comprising continuously applying an antigen epicutaneously to an area of the skin of the subject under conditions sufficient to induce (CTLA-4+, CD4+, CD25+)Foxp3+ Treg cells and to modulate CpG island methylation of transcription factor DNA.

A further object of the invention relates to an antigen, or a composition comprising an antigen, for use to treat or prevent a proliferative, autoimmune, allergic or inflammatory disease in a subject by continuously applying said antigen or composition epicutaneously to an area of the skin of the subject, said application leading to a prevention or treatment of said disease. The application is preferably performed under conditions sufficient to induce (CTLA-4+, CD4+, CD25+) Foxp3+ Treg cells and/or to modulate CpG island methylation of transcription factor DNA.

A further object of the invention relates to a method for inducing (CTLA-4+, CD4+, CD25+)Foxp3+ Treg cells in a subject, the method comprising applying an antigen epicutaneously to an area of the skin of the subject. The invention also relates to an antigen for use for inducing (CTLA-4+, CD4+, CD25+)Foxp3+ Treg cells in a subject by applying said antigen epicutaneously to an area of the skin of the subject. In a preferred embodiment, the Treg cells are naïve (CD44lo/CD62L+) Foxp3+ Tregs or effector (CD44hi/CD62L−) Foxp3+ Tregs. As will be discussed, these cells exhibit particular biological activities allowing strong and persistent immunorebalancing in a subject.

A further object of the invention relates to a method for inducing LAP+ Treg cells in a subject, the method comprising applying an antigen epicutaneously to an area of the skin of the subject. The invention also relates to an antigen for use for LAP+ Treg cells in a subject by applying said antigen epicutaneously to an area of the skin of the subject. As will be discussed, these cells exhibit particular biological activities allowing strong and persistent immunorebalancing in a subject.

A further object of the invention relates to an antigen for use as a medicament for inducing (CTLA-4+, CD4+, CD25+)Foxp3+ Treg cells and/or LAP+ Treg cells in a subject by applying said antigen epicutaneously to an area of the skin of the subject.

The invention also relates to a method for producing (CTLA-4+, CD4+, CD25+)Foxp3+ Treg cells and/or LAP+ Treg cells, the method comprising (i) stimulating (CTLA-4+, CD4+, CD25+)Foxp3+ Tregs and/or LAP+ Tregs in a mammal by applying an antigen epicutaneously to an area of the skin of the mammal, (ii) collecting (CTLA-4+, CD4+, CD25+)Foxp3+ Tregs and/or LAP+ Tregs, respectively, from said mammal, and (iii) culturing or expanding or preserving or formulating the (CTLA-4+, CD4+, CD25+)Foxp3+ Tregs and/or LAP+ Tregs, respectively.

The antigen for use in the present compositions or methods may be of different nature, such as preferably a protein, peptide, nucleic acid, lipid, particle, metal, or a combination thereof. In a preferred embodiment, the antigen is an antigen to which the subject exhibits natural or induced skin sensitivity.

The invention may be used in any mammalian subject, preferably in human subjects, including children and adults.

### LEGEND TO THE FIGURES

FIG. 1: Analysis of Tregs induced by 8 weeks of EPIT in mice sensitized to peanut. Spleen cells of naïve, sensitized not treated (Sham) or sensitized EPIT treated mice were stained with anti-mouse CD4, CD25, Foxp3, IL-10 and the percentages of CD4+CD25+Foxp3+, CD4+CD25+IL-10+, and CD4+LAP+ cells were measured by flow cytometry.

FIG. 2: Analysis of induction of CTLA-4 expression in Tregs induced by EPIT in mice sensitized to peanut. Spleen cells of naïve, Sham or EPIT mice were stained with anti-mouse CD4, CD25, Foxp3, and CTLA-4. Among the lymphocyte identified by FSC/SSC, cells were gated on CD4+CD25+Foxp3+ and the percentage of cells expressing CTLA-4 was analyzed.

FIG. 3: Decrease of peanut-specific TH2 cytokine production by splenocytes from EPIT is blocked by anti-CTLA-4 but not anti-IL-10. Spleen cells of naïve, Sham or EPIT mice were cultured in medium alone, medium+peanut protein extract (PPE), medium+PPE in presence of anti-IL-10 blocking antibody or medium+PPE in presence of anti-CTLA-4 blocking antibody for 3 days. Supernatants were then harvested and IL-5 and IL-13 were measured by ELISA.

FIG. 4: Analysis of effector (CD44hiCD62L−) or naïve (CD44loCD62L+) phentotype of Tregs induced by EPIT in mice sensitized to peanut. Spleen cells of naïve, Sham or EPIT mice were stained with anti-mouse CD4, CD25, Foxp3, CD44 and CD62L. Cells were gated on CD4+ among the lymphocyte identified by FSC/SSC, and the percentages of CD25+Foxp3+CD44hiCD62L− (effector Tregs) or CD25+Foxp3+CD44loCD62L+(Naïve Tregs) were analyzed by flow cytometry.

FIG. 5: EPIT increased both natural (CD304+) and induced (CD304−) Tregs in mice sensitized to peanut. Spleen cells of naïve, Sham or EPIT mice were stained with anti-mouse CD4, CD25, Foxp3, and CD304. Cells were gated on CD4+ among the lymphocyte identified by FSC/SSC, the percentages of CD25+Foxp3+CD304+(nTregs) or CD25+Foxp3+CD304− (iTregs) were analyzed by flow cytometry.

FIG. 6: EPIT induced a large repertoire of homing receptors on Tregs. Spleen cells of naïve, Sham or EPIT mice were stained with anti-mouse CD4, CD25, Foxp3, and CCR4, Cutaneous Lymphocyte Antigen (CLA), CCR9, CXCR3, CCR6, CCR8 or CCR3. Co-expression of CLA and CCR9 was analyzed. Among the lymphocyte identified by FSC/SSC, cells were gated on CD4+CD25+Foxp3+ and the expression of homing receptors were analyzed.

FIG. 7: DNA analysis 8 weeks after the end of EPIT. Measurement of methylation level in the promoter region of GATA-3 by using commercial kit (SA biosciences) based on detection of remaining input DNA after cleavage with a methylation-sensitive and/or a methylation-dependent restriction enzyme.

FIG. 8: DNA analysis in whole spleen during EPIT. Measurement of methylation level in the promoter region of GATA-3 by using commercial kit (SA biosciences) based on detection of remaining input DNA after cleavage with a methylation-sensitive and/or a methylation-dependent restriction enzyme

FIG. 9: DNA analysis in whole blood during EPIT. Measurement of methylation level in the promoter region of GATA-3 by using commercial kit (SA biosciences) based on detection of remaining input DNA after cleavage with a methylation-sensitive and/or a methylation-dependent restriction enzyme

FIG. 10: Analysis of the methylation levels of GATA-3 gene in CD4 cells isolated from (a) spleen and (b) whole blood at week 1 (1 w), week 2 (2 w), week 4 (4 w), week 6 (6 w), week 8 (8 w) of EPIT® and 8 weeks after the end of EPIT® (8+8 w). Results are expressed as mean±SD. *, p<0.05, ** p<0.01.

FIG. 11: Analysis of the methylation levels of Foxp3 gene in CD4 cells isolated from (a) spleen and (b) whole blood at week 1 (1 w), week 2 (2 w), week 4 (4 w), week 6 (6 w), week 8 (8 w) of EPIT® and 8 weeks after the end of EPIT® (8+8 w). Results are expressed as mean±SD. **, p<0.01, *** p<0.001.

### DETAILED DESCRIPTION OF THE INVENTION

The present invention stems from the unexpected finding that continuous epicutaneous treatment of a subject can induce particular subsets of regulatory T cells, distinct from the cells observed in other immunotherapies, suitable to cause a substantial and long-lasting improvement in a subject condition. The invention may be used in a preventive context, to improve the immunobalance of a subject and avoid the onset or development of diseases, as well as in a therapeutic context, to improve a subject recovery. The invention is particularly suitable to prevent or treat a proliferative, autoimmune, allergic or inflammatory disease in any mammalian subject, preferably in human subjects.

The present disclosure will be best understood by reference to the following definitions:

### DEFINITIONS

“Epicutaneous” administration refers to the application of a substance (i.e., an antigen) on the skin of the subject under conditions allowing a contact with the surface of the skin. Epicutaneous application is preferably performed without any skin perforation or pre-treatment leading to a significant change in skin structure. Skin application is preferably maintained in conditions allowing penetration of the allergen in the superficial layer(s) of the skin and/or and for a period of time sufficient to allow contact of the allergen with immune cells. Epicutaneous administration is preferably performed with a skin device, such as a patch.

The term “continuous”, in relation to the epicutaneous application, designates an application that results in a long-lasting, preferably a permanent contact of the antigen with the immune system or in the long-lasting, preferably permanent, presence of immune cells generated by the contact with said antigen. The continuous application is preferably from 3 to up to 60 months. The continuous application shall more preferably ensure a daily contact of the antigen with the immune system.

Within the context of this invention, the term “preventing” is meant to include protecting the subject from onset or development of a disease, delaying appearance or occurrence of such disease, or inhibiting or reducing the magnitude of any such disease. Within the context of the present invention, the term “intact skin” indicates that the integrity of the stratum corneum layer should be substantially maintained. For the performance of the invention, it is indeed most preferred to apply antigens on intact skin, e.g., on a surface or portion of the skin where the integrity of the stratum corneum is essentially maintained. By maintaining this integrity, the response obtained is highly oriented in the sense of immune tolerance. Accordingly, although gentle cleaning of the skin surface may be performed at the site of application, e.g., hydration, water cleaning, or very gentle stripping (4 or 5 tape stripping at most), to remove e.g., corneocytes, the skin should not be pre-treated, thus maintaining substantial integrity of the stratum corneum. In particular, strong abrasion of the skin should not be performed, since such pre-treatments disrupt, or remove all or part of the stratum corneum. Similarly, perforation of the stratum corneum should be avoided.

The term “antigen” refers to an immunologic molecule involved in an immune reaction. The antigen may be of various nature, such as a lipid, protein, peptide, polypeptide, nucleic acid, metal, plastic, etc. In a particular embodiment, the antigen is a protein, polypeptide and/or peptide. The antigen may be in a natural state, or produced artificially (e.g., by recombinant and/or enzymatic techniques for instance). The antigen may be structurally altered or modified to improve its stability, immunogenicity, etc. The antigen may be pure or in admixture with other constituents. The antigen may also be a mixture of several molecules (e.g., an extract). As will be discussed further below, the antigen may be used in different states, such as liquid or dry.

### Methods for Inducing Particular CTLA-4+ Treg Subsets & Gene Methylation

The present invention discloses the unexpected finding that epicutaneous administration of an antigen can induce or stimulate non antigen-specific CTLA-4+ Tregs in a subject. Such particular cells exhibit remarkable properties that improve a subject condition by improving the immunobalance in said subject.

Tregs are a class of T cells having immunosuppressive or immunoregulatory functions. Various populations of regulatory T (Treg) cells have been shown to play a central role in the maintenance of peripheral immune homeostasis and the establishment of controlled immune responses. Both naturally occurring CD4+CD25+ Treg cells and inducible populations of allergen-specific, IL-10-secreting Treg type 1 (Tr1), TGFβ-secreting Treg (Th3) cells inhibit allergen-specific effector cells in experimental models. The suppressive capacity of Tr1 cells is IL-10-dependent whereas CD4+CD25+Foxp3+ Tregs mediate suppression by cell-cell contact. Skewing of allergen-specific effector T cells to a regulatory phenotype appears to be a key event in the development of healthy immune response to antigens and successful outcome in immunotherapy.

Forkhead box protein 3-positive CD4+CD25+ Treg cells, Th3 and Tr1 cells contribute to the control of antigen-specific immune responses in several major ways, which can be summarized as suppression of dendritic cells that support the generation of effector T cells; suppression of effector Th1, Th2, and Th17 cells; suppression of allergen-specific IgE and induction of IgG4; suppression of mast cells, basophils, and eosinophils; interaction with resident tissue cells and remodeling; and suppression of effector T-cell migration to tissues. Depending on tissues, origin and stimulatory conditions, the different subsets differ in their cytokine production and surface markers expression, and how they suppress immune responses.

It is important to note that the mechanisms of suppression can singly account for Treg cell-mediated control of immunity. Moreover, the Foxp3-dependent suppressor program implemented by Treg cells keeps in check various types of effector immune responses to self-antigens and pathogens. In the past few years, mounting experimental evidence has suggested that distinct suppressor mechanisms prominently feature in particular tissue and inflammatory settings. For example, the expression of Tbet, a key transcription factor in Th1 effector cell differentiation in Treg cells enables them through the expression of CXCR3 to migrate, proliferate and accumulate at the site of Th1 responses (Josefowicz et al, 2012, Annu. Rev. Immunol. 2012. 30:531-64, Regulatory T Cells: Mechanisms of Differentiation and Function).

Bohle B, et al. (The Journal of allergy and clinical immunology. 2007; 120(3):707-13), Francis J N, et al (Journal of allergy and clinical immunology. 2008; 121(5):1120-5) and Mobs C, et al (The e2. Epub 2008/04/01) have suggested that sublingual or subcutaneous immunotherapies can induce IL-10-producing T regulatory cells. Surprisingly, the invention shows that epicutaneous immunotherapy can induce a particular subset of Tregs, designated cell-contact Foxp3+ Treg cells as expressing CTLA 4+ or other cell surface markers (as LAP), which are distinct from the cells induced by other immunotherapies acting via the secretion of cytokines (IL-10). These cells, contrary to IL-10 producing Treg cells, essentially act by cell contacts, to alter the activity of antigen-presenting cells, B cells and mast cells. Through such mechanism, these cells are able not only to affect antigen-specific immunity, but more generally to restore a proper immunobalance in a subject. By continuous epicutaneous application of an antigen, it is therefore possible to induce or stimulate such cell population in a subject, leading to a preventive and curative approach for the treatment of various pathological conditions. Furthermore, the invention also surprisingly shows that such continuous epicutaneous treatment also causes epigenetic modifications in particular genes, which further induce long-lasting immunorebalancing. More specifically, the results show that the method of this invention increases CpG island methylation of the GATA-3 gene. The GATA-3 gene is a transcription factor involved in immune cell activity. By increasing the methylation of this gene, the method of the invention causes an inhibition of this transcription factor, modulates the expression of Th2 transcription factors, leading to a sustained immunorebalancing.

An object of the invention thus resides in a method for improving a subject condition, the method comprising continuously applying an antigen epicutaneously to an area of the skin of the subject. More preferably, the method comprises the continuous epicutaneous application of the antigen under conditions sufficient to induce (CTLA-4+, CD4+, CD25+) Foxp3+ Treg cells and/or to increase CpG island methylation of the GATA-3 gene.

A further object of the invention relates to a method for preventing or reducing the onset or progression of a proliferative, autoimmune, allergic or inflammatory disease in a subject, comprising continuously applying an antigen epicutaneously to an area of the skin of the subject, said application leading to a prevention or reduction of said disease. The application is preferably performed under conditions sufficient to induce (CTLA-4+, CD4+, CD25+) Foxp3+ Treg cells and/or to increase CpG island methylation of the GATA-3 gene.

A further particular object of the invention relates to a method for treating or alleviating an existing proliferative, autoimmune, allergic or inflammatory disease in a subject, comprising continuously applying an antigen epicutaneously to an area of the skin of the subject, said application leading to a treatment or alleviation of said disease. The application is preferably performed under conditions sufficient to induce (CTLA-4+, CD4+, CD25+) Foxp3+ Treg cells and/or to increase CpG island methylation of the GATA-3 gene.

A further object of the invention relates to an antigen, or a composition comprising an antigen, for use to treat or prevent a proliferative, autoimmune, allergic or inflammatory disease in a subject by continuously applying said antigen or composition epicutaneously to an area of the skin of the subject, said application leading to a prevention or treatment of said disease. The application is preferably performed under conditions sufficient to induce (CTLA-4+, CD4+, CD25+) Foxp3+ Treg cells and/or LAP+ Treg cells, and/or to increase CpG island methylation of the GATA-3 gene and/or to decrease CpG island methylation of Foxp3 gene, more preferably under conditions sufficient to induce (CTLA-4+, CD4+, CD25+) Foxp3+ Treg cells and/or to increase CpG island methylation of the GATA-3 gene concomitant to decrease CpG island methylation of Foxp3 gene.

A further object of the invention relates to a method for inducing (CTLA-4+, CD4+, CD25+)Foxp3+ Treg cells in a subject, the method comprising applying an antigen epicutaneously to an area of the skin of the subject. The invention also relates to an antigen for use for inducing (CTLA-4+, CD4+, CD25+)Foxp3+ Treg cells in a subject by applying said antigen epicutaneously to an area of the skin of the subject.

A further object of the invention relates to an antigen, or a composition comprising an antigen, for use to treat or prevent a proliferative, autoimmune, allergic or inflammatory disease in a subject, said diseases resulting from a tolerance breakdown, by continuously applying said antigen or composition epicutaneously to an area of the skin of the subject, said application leading to tolerance restoring. The application is preferably performed under conditions sufficient to induce (CTLA-4+, CD4+, CD25+) Foxp3+ Treg cells and/or to increase CpG island methylation of the GATA-3 gene and/or to (e.g., concomitantly) decrease CpG island methylation of Foxp3 gene.

Advantageously, the induced Treg cells can be both naïve (CD44lo/CD62L+) Foxp3+ Tregs or effector (CD44hi/CD62L−) Foxp3+ Tregs. Furthermore, advantageously, the method also induces CD304-Treg cells, further strengthening the immune system in the subject. In addition, as illustrated in the examples, the repertoire of (CTLA-4+, CD4+, CD25+)Foxp3+ Treg cells induced is diverse since these cells may further express CCR8, CCR9, Cutaneous Lymphocyte Antigen (CLA), CCR6, CCR4 and/or CXCR3 molecules. By inducing such a diverse repertoire in CTLA-4+ cells, the method of the invention provides strong protection and defense against disease conditions in a subject.

Natural Tregs are crucial in suppressing effector T cells in the draining lymph nodes and in inhibiting tissue inflammation in the target organ. Although all nTreg cells express the transcription factor Foxp3, it has become clear that these Foxp3 Treg cells further specialize, express defined transcription factor factors and suppress distinct subsets of effector T cells. As Treg induce tolerance and inhibit tissue inflammation, it is perhaps not surprising that they also specialize in the tissue niches. A recently identified example of tissue-specific Treg is adipose tissue resident Foxp3+ Tregs found in fat and acquiring a distinct molecular signature after trafficking to the adipose tissue. Another example of tissue specific Tregs are mucosal IL-10 secreting Tr1 cells that do not express Foxp3 but are activated by intestinal bacteria and suppress Th1 and Th17 activated intestinal inflammation.

The antigen used in the method may be any antigen to which the subject is sensitive. In particular, if sensitivity to an antigen is observed in a subject, the method of the invention can be implemented using such an antigen, by continuous application.

Alternatively, auto-antigens may be used, especially for treating or alleviating autoimmune disorders.

In another embodiment, sensitivity to an antigen may be induced in a subject, by first exposing the subject to said antigen. Subsequently, a strong immuno-rebalancing in said subject can be induced by continuous epicutaneous application thereof.

In a further alternative, a “universal” antigen may be used, e.g., an antigen that causes a response in any subject. Examples of such antigens include for instance certain metals.

Accordingly, upon detection of a first antigenic (e.g., allergic reaction) in a subject, the continuous epicutaneous treatment of the invention can be started. The antigenic response detected in said subject may be an allergy (e.g., a food allergy or a dust mite allergy), an autoimmune disease, an inflammatory reaction, etc. By administering epicutaneously and continuously to these subjects a suitable amount of the antigen, it is possible to effectively improve the subject condition by inducing preferred Treg cells.

It is preferred to perform the treatment shortly after detection or verification of the presence of an antigenic sensitivity in the subject. Indeed, by acting early, it is possible to induce a strong and potent immune response which is expected to prevent aggravation or development of diseases.

Detection or verification of an antigenic response can be performed using techniques per se known in the art. Examples of methods suitable include the use of a prick-test, the dosage of IgE, an atopy patch-test, the detection of an autoimmune disease, the detection of an immune disease, the detection of an allergy or proliferative cell disorder. The detection of an antigenic reaction includes the detection of a sensitivity to an antigen, even if no clinical sign of the disease are present. Generally, the detection comes first from appearance of classical disease symptoms (inflammation, swallowing, etc). It may then be tested again, or verified, e.g., by a practitioner, using any of the above techniques, if needed. Once an antigenic sensitivity has been detected or verified for a subject, treatment of the invention may start. The treatment efficacy will increase if the treatment is started shortly (e.g., immediately or within weeks, for example less than 4 weeks) after detection or verification of the antigenic response. However, treatment is also potent in subjects showing several antigenic reactions or an advanced disease.

In accordance with one aspect of the invention, a protective method is provided comprising epicutaneous application of at least one antigen to the subject. Most preferably, the at least one antigen applied is an antigen to which the subject exhibits natural or induced sensitivity.

The epicutaneous treatment protocol may be adjusted by the practitioner. Typically, the allergen is applied continuously for a period of time sufficient to induce the CTLA-4+ Tregs and/or GATA-3 methylation or Foxp3 demethylation. In a preferred embodiment, the treatment is performed during a period of at least 3 months, preferably at least 6 months, and more preferably between 6 and 60 months. During this treatment period, the antigen may be applied at various frequencies, such as weekly, every other day, or daily. The dose of antigen used for each application can be adjusted by the skilled artisan. Typically, it is comprised between 0.1 and 10000 μg, preferably between 20 and 1000 μg. The improvement in the subject can be verified at any time by conventional examination. In particular, the existence of an improvement in the treated subjects can be verified by a decrease in, or a disappearance or absence of clinical signs of disease. Dosing immune cells or mediators in the subject may also be performed, although the absence of clinical signs is sufficient. Once the proper response has been generated, it is possible to reduce the treatment (dosage, frequency of application, time of application), or to even stop it. As indicated above, the treatment is typically to be maintained for a period of time of at least 3 months preferably at least 6 months, and more preferably between 6 and 36 months, with repeated applications at different intervals: each day or each other day, or each week in order to continuously stimulate the immune system of the patient.

Depending on the disease, the antigen used may be different. For autoimmune disorders, a portion of the auto-antigen may be used.

As stated before, a universal antigen may also be used, applicable to all of these pathological conditions, to improve the subject immunobalance.

In a preferred embodiment, the antigen is applied without adjuvant. However, although not preferred, the antigen may be combined with an adjuvant, i.e., any substance that e.g., activates or accelerates the immune system to cause an enhanced immune response. Examples of adjuvants include mineral salts, such as calcium phosphate, aluminium phosphate and aluminium hydroxide; immunostimulatory DNA or RNA, such as CpG oligonucleotides; proteins, such as antibodies or Toll-like receptor binding proteins; saponins e.g. QS21; cytokines; muramyl dipeptide derivatives; LPS; MPL and derivatives including 3D-MPL; GM-CSF (Granulocyte-macrophage colony-stimulating factor); retinoic acid; imiquimod; colloidal particles; complete or incomplete Freund's adjuvant; Ribi's adjuvant or bacterial toxin e.g. cholera toxin or enterotoxin (LT).

In a further preferred embodiment, the antigen is applied on an intact area of the skin.

It is particularly preferred to apply the antigen without adjuvant and on intact skin.

The antigen may be applied using different techniques or devices suitable to maintain contact between the antigen and the skin of the subject. Such devices include, without limitation, a patch, a tape, a dressing, a sheet, or any other form known to those skilled in the art. Preferably, the skin device is a patch, even more preferably an occlusive patch. Preferred patch devices do not alter integrity of the skin, i.e., they are non-perforating. In the most preferred embodiment, the method of the invention uses a skin patch device as described in international patent applications WO2002/071950 and WO 2007/122226. Such a device is occlusive and is configured to use an antigen in dry form, the antigen being maintained on the patch through electrostatic and/or Van der Waals forces, with no added adhesive. The preparation and characteristics of such a device (termed Viaskin®) are disclosed in detail in the above identified applications, which are incorporated herein by reference in their entireties.

For the performance of the present invention, it is particularly well suited to use a device comprising a backing adapted to create with the skin a hermetically closed chamber, this backing having on its skin facing side within the chamber the dry antigen adhered through electrostatic forces and/or Van der Waals forces. Upon application to the skin, moisture increases in the chamber, leading to antigen dissolution and contacting with the skin.

In another preferred embodiment of the invention, the antigen is applied on the skin of the subject using an occlusive patch device comprising a support to which the antigen is bound. Preferably, the antigen is bound to the support of the patch through electrostatic or Van der Waals forces, with no added adhesive. In particular embodiments, the support of the patch may comprise glass or a polymer chosen from the group consisting of cellulose plastics (CA, CP), polyvinyl chloride (PVC), polypropylenes, polystyrenes, polyurethanes, polycarbonates, polyacrylics, polyolefines, polyesters, polyethylenes and ethylene-vinyl acrylates (EVA). The patch may be secured to the skin by an adhesive border.

In a most preferred embodiment, the method is performed using a dry antigen preparation, which is preferably applied on the skin using an electrostatic skin device. In this regard, the term “dry” designates the fact that the antigen is substantially powdered, e.g., in the form of particles which may be individualized or agglomerated.

Although less preferred, the antigen may be in liquid form and applied using known devices, such as occlusive devices having a reservoir or a perforated membrane.

The invention may be suitable for improving the condition of a subject in general, by improving the immunobalance in the subject. Such treated subjects would be less prone to developing diseases.

The invention may also be used to prevent or treat specific diseases, particularly a proliferative, (auto) immune, or inflammatory disease.

Examples of allergic diseases include allergies, asthma.

Examples of (auto) immune diseases, include diabetes, allograft rejection, Crohn's disease, RA, MS.

Examples of proliferative diseases include cancers.

Examples of inflammatory diseases include Crohn' disease, MS.

Treating shall particularly include an improvement in the symptoms, life expectancy, a reduction in severity or pain, a blocking or reversion of cause of disease. The term treatment includes the breaking of a peripheral or central tolerance that causes or contributed to a pathological condition.

The invention is particularly suited to treat or alleviate type I diabetes.

Further aspects and advantages of the invention will be disclosed in the following experimental section, which should be considered as illustrative.

### EXAMPLES

### Example 1

### Epicutaneous Immunotherapy (EPIT) Induces CTLA-4+ Regulatory T Cells (Tregs)

### A. Materials and Methods

Twenty BALB/c mice were sensitized orally to peanut protein extract with cholera toxin. After sensitization 10 mice were continuously treated for 8 weeks by EPIT or not treated (Sham). Ten naïve mice were included as control. After treatment period, mice were killed and spleen cells were isolated for immunostaining and flow cytometry analysis or in vitro restimulation.

Splenocytes were stained with anti-mouse CD4, CD25, Foxp3, IL-10, CTLA-4, LAP antibodies. Cells were gated on CD4+ among the lymphocyte identified by FSC/SSC, the percentages of LAP+, CD25+Foxp3+ or CD25+IL10+ were analyzed. Among the lymphocyte identified by FSC/SSC, cells were gated on CD4+CD25+Foxp3+ and the percentage of cells expressing CTLA-4 was analyzed.

Spleen cells were restimulated by peanut protein extract alone or with anti-IL10 or anti-CTLA-4 blocking antibodies for 3 days. Cells without stimulation were used as control. Cell supernatants were then tested for the presence of IL-5 or IL-13 by ELISA.

Experiment was reproduced twice.

### B. Results

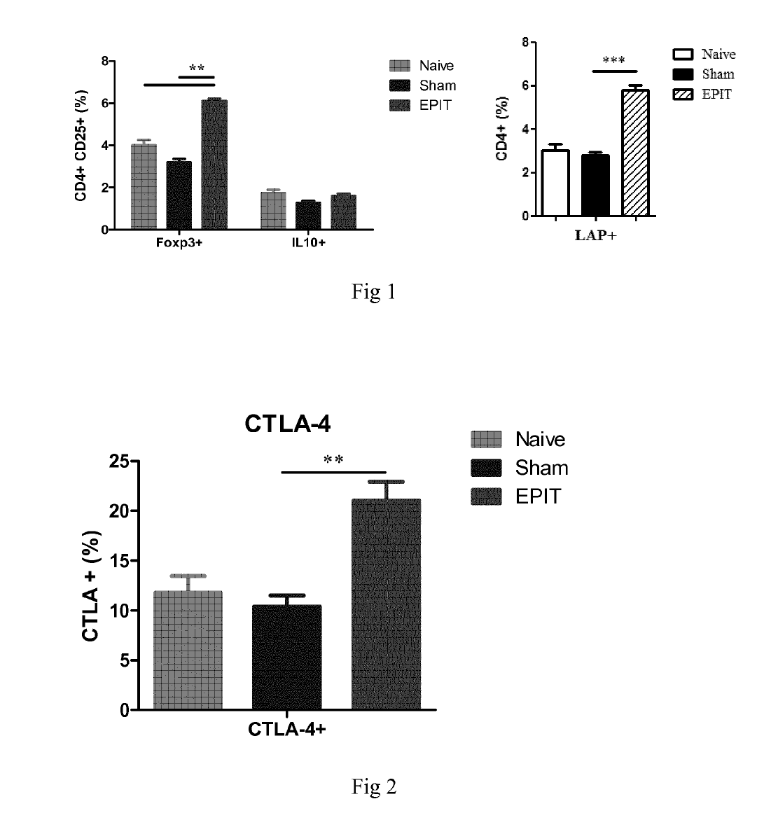

Peanut sensitized mice were treated by EPIT for 8 weeks. EPIT increased CD4+CD25+Foxp3+ cells and CD4+LAP+ cells but not CD4+CD25+IL-10+ cells (FIG. 1). Furthermore, the phenotyping showed that a high proportion of Treg cells induced by continuous EPIT are CTLA-4+ cells (FIG. 2).

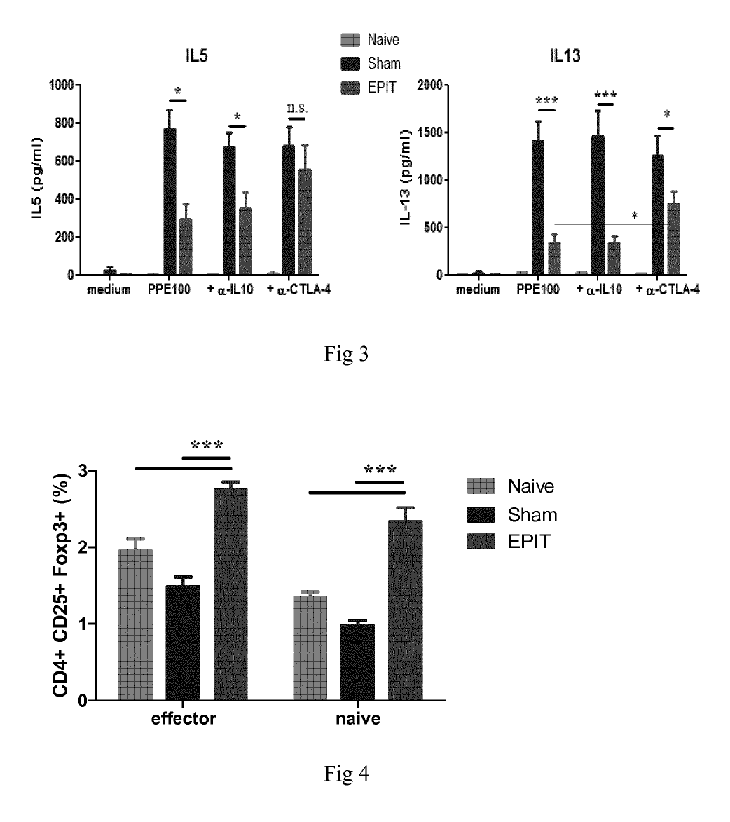

Conventional specific immunotherapy increases the frequency of IL-10+ regulatory cells which mediate their suppressive activity by secretion of interleukin-10 (IL-10). By contrast, suppressive acitivity of Tregs induced by continuous EPIT is not mediated by IL-10 but towards CTLA-4. The suppressive capacity is analyzed by the decrease of secretion of Th2 cytokine in continuous EPIT compared to Sham. This suppressive activity is still observed when we blocked the IL-10 pathway but is inhibited when we blocked the CTLA-4 pathway (FIG. 3).

### Example 2

### Continuous EPIT Increases Naïve and Effector Tregs

### A. Materials and Methods

Twenty BALB/c mice were sensitized orally to peanut protein extract with cholera toxin. After sensitization 10 mice were continuously treated for 8 weeks by EPIT or not treated (Sham). Ten naïve mice were included as control. After treatment period, mice were killed and spleen cells were isolated for immunostaining and flow cytometry analysis.

Cells were gated on CD4+ among the lymphocyte identified by FSC/SSC, the percentages of CD25+Foxp3+CD44hiCD62L− (effector Tregs) or CD25+Foxp3+CD44loCD62L+ (Naïve Tregs) were analyzed.

Experiment was reproduced twice.

### B. Results

The Foxp3+ Tregs induced by EPIT present a particular phenotype: After EPIT, both naive (CD44lo/CD62L+) and effector (CD44hi/CD62−) Foxp3 Tregs increased significantly (FIG. 4).

### Example 3

### Continuous EPIT Increases Natural and Induced Tregs

### A. Materials and Methods

Twenty BALB/c mice were sensitized orally to peanut protein extract with cholera toxin. After sensitization 10 mice were continuously treated for 8 weeks by EPIT or not treated (Sham). Ten naïve mice were included as control. After treatment period, mice were killed and spleen cells were isolated for immunostaining and flow cytometry analysis.

Cells were gated on CD4+ among the lymphocyte identified by FSC/SSC, the percentages of CD25+Foxp3+CD304+(nTregs) or CD25+Foxp3+CD304− (iTregs) were analyzed.

Experiment was reproduced twice.

### B. Results

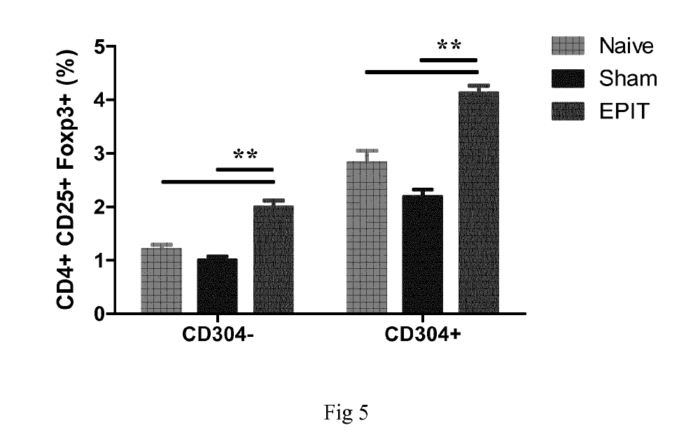

After EPIT, both natural (CD304+) and induced (CD304−) Foxp3 Tregs increased significantly (FIG. 5).

### Example 4

### Continuous EPIT Induce Diverse CTLA-4+ Treg Cells

### A. Materials and Methods

Sixteen BALB/c mice were sensitized orally to peanut protein extract with cholera toxin. After sensitization 8 mice were continuously treated for 8 weeks by EPIT or not treated (Sham). Eight naïve mice were included as control. After treatment period, mice were killed and spleen cells were isolated for immunostaining and flow cytometry analysis.

Among the lymphocyte identified by FSC/SSC, cells were gated on CD4+CD25+Foxp3+ and the expression of CCR4 (lung homing receptor), Cutaneous Lymphocyte antigen (CLA) (skin homing receptor), CCR9 (gut homing receptor), CXCR3 (TH1 inflammation homing receptor), CCR6 (Th17 inflammation homing receptor), CCR8 (Th2 inflammation homing receptor) and CCR3 (eosinophil homing receptor) were analyzed.

Experiment was reproduced twice.

### B. Results

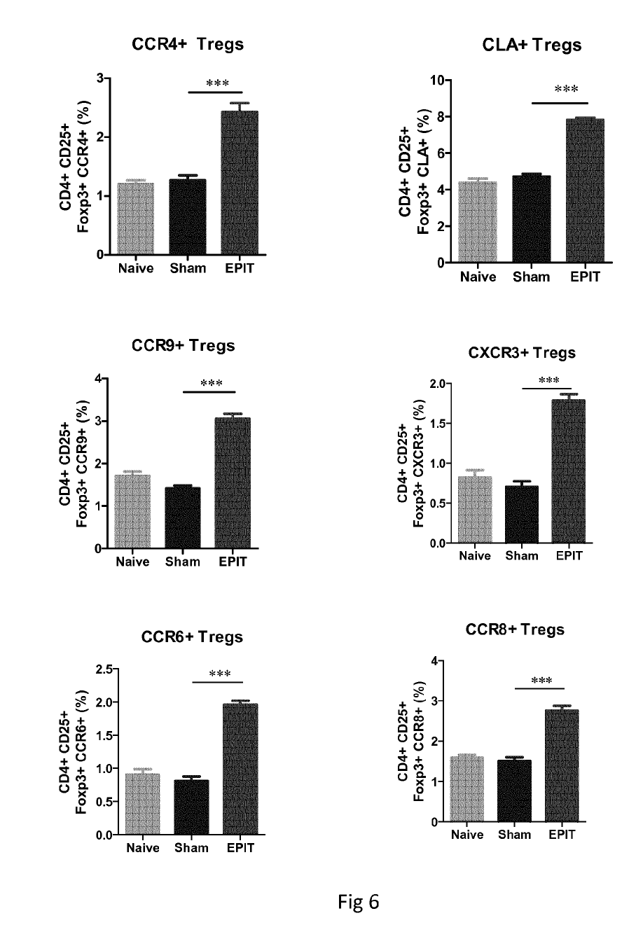

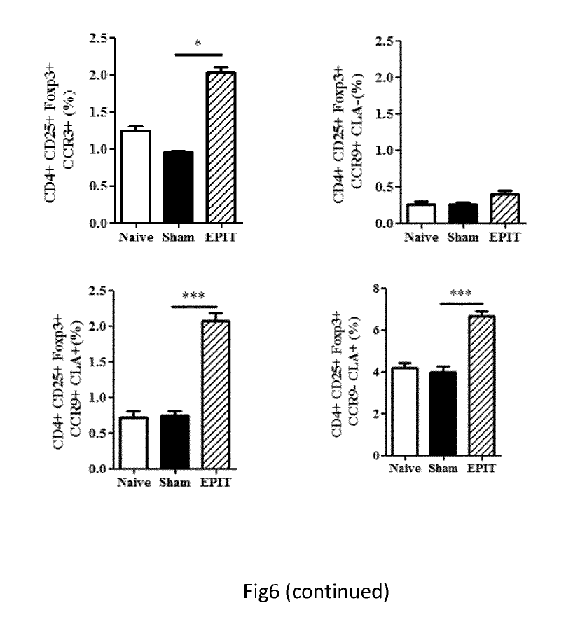

After EPIT, homing receptor expression on Foxp3 Tregs is diverse, suggesting a capacity to migrate to diverse organs and then protect from antigen exposure from different routes. Tregs induced by EPIT expressed high level of CCR4 (lung homing receptor), Cutaneous Lymphocyte antigen (CLA) (skin homing receptor), CCR9 (gut homing receptor), CXCR3 (TH1 inflammation homing receptor), CCR6 (Th17 inflammation homing receptor), CCR8 (Th2 inflammation homing receptor) and CCR3 (eosinophil homing receptor) (FIG. 6).

Different subsets of Tregs have been described, the 3 most relevant classes being the IL-10-producing Tr1 cells, the TGF-β-producing Th3 cells (LAP+), and the CD4+CD25+ Tregs. Depending on the tissue, origin and stimulatory condition, the different subsets differ in their cytokine production and surface markers expression, and on how they can suppress immune responses. In our model, EPIT induced significant increase of Foxp3+ Tregs and LAP+ Tregs. The suppressive activity of EPIT-induced Tregs did not depend on IL-10 but needs CTLA-4.

EPIT-induced Tregs were able to protect sensitized mice from esophagus inflammation following peanut oral exposure. This could result from the expression of CCR3, receptor of eotaxin, by EPIT-induced Tregs.

The presence of increased level of CLA+CCR9+ Tregs in iLN, but also in spleen and mLN after EPIT clearly suggests that EPIT induces Tregs, in skin or in draining lymph nodes after Langerhans cell migration. Part of these Tregs also expressed CCR9 and were then able to migrate toward the mLN and gut mucosa. The wider range of homing receptor expressed by EPIT-induced Tregs suggests that EPIT-induced Tregs are able to migrate to various sites of allergen exposure and to induce protection from Th2-induced inflammation and suppress local response to allergen stimulation, thus inducing a global tolerance rather than a local desensitization.

### Example 5

### Continuous EPIT Induces DNA Methylation

### A. Materials and Methods

Sixty BALB/c mice were orally sensitized to milk and then treated by continuous epicutaneous immunotherapy (EPIT) or not desensitized (Sham). Mice were killed immediately or 8 weeks after the end of treatment. In another set of experiment, mice were sensitized to peanuts and divided into the same groups for treatment (EPIT, Sham). Ten naïve mice were also included in the study. DNA methylation was analyzed in spleen samples taken for all mice at each sacrifice.

### B. Results

Epigenetic control is a well-established means of gene regulation within the immune system. Mechanism such as histone modifications and DNA methylation carefully govern cell fate decisions in developing lymphocytes. We have therefore investigated whether the protective effect induced by continuous EPIT could involve epigenetic modifications, particularly for transcription factors.

In a model of sensitized mice, we investigated if continuous EPIT induced epigenetic modifications and compared to sublingual immunotherapy, and more focused on GATA-3 and Tbet as transcription factors.

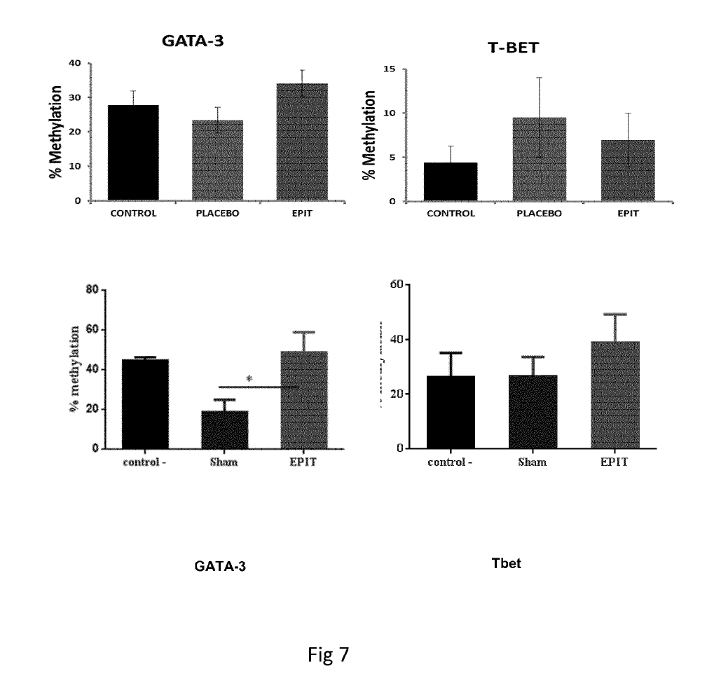

Continuous EPIT significantly increased methylation in the CpG islands of GATA-3 versus Sham (FIG. 7). This modification was maintained 2 months after the end of immunotherapy (FIG. 7) thus validating the necessity of a continuous application of the antigen.

In another set of experiment, 60 BALB/c mice were sensitized to peanuts and 30 of them were treated by EPIT. At different time points during EPIT (1, 2, 4, 6, 8 weeks), the increase of methylation of GATA-3 promoters was evaluated in whole spleen cells and whole blood.

The implementation of epigenetic modifications by EPIT was continued at cell levels. CD4, CD8 and B cells were sorted from blood and spleen (at weeks 1, 2, 4, 6, 8 and 8 weeks after the end of treatment) by using magnetic beads coupled to specific antibodies. DNA was extracted from sorted cells and methylation analysed by bisulfite treatment followed by pyrosequencing.

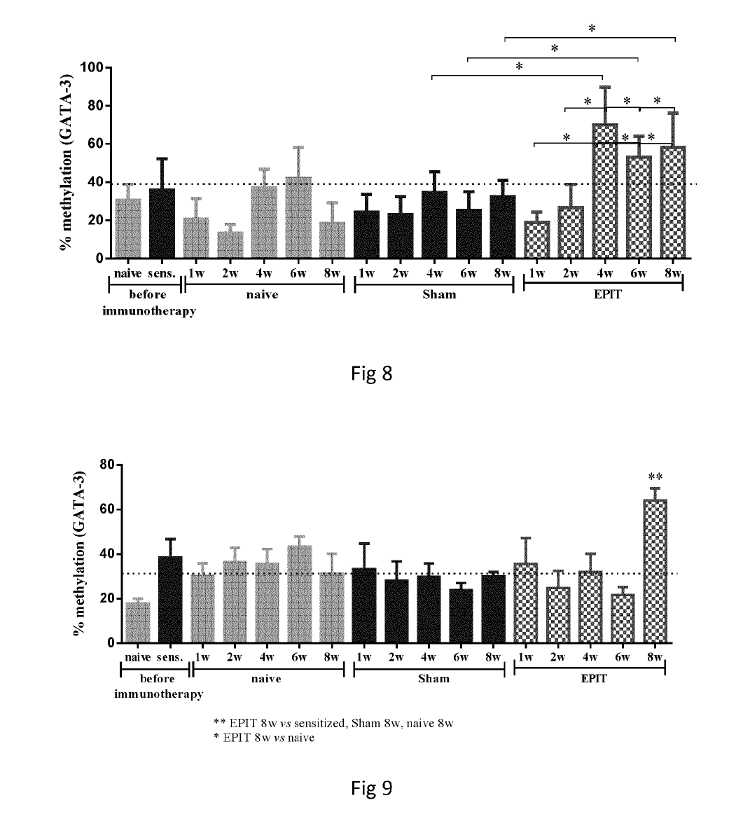

As shown in FIGS. 8 and 9, the methylation level was increased from the 4^{th} week to the end of EPIT in the spleen whereas the same parameter was increased in blood after 8 weeks of EPIT.

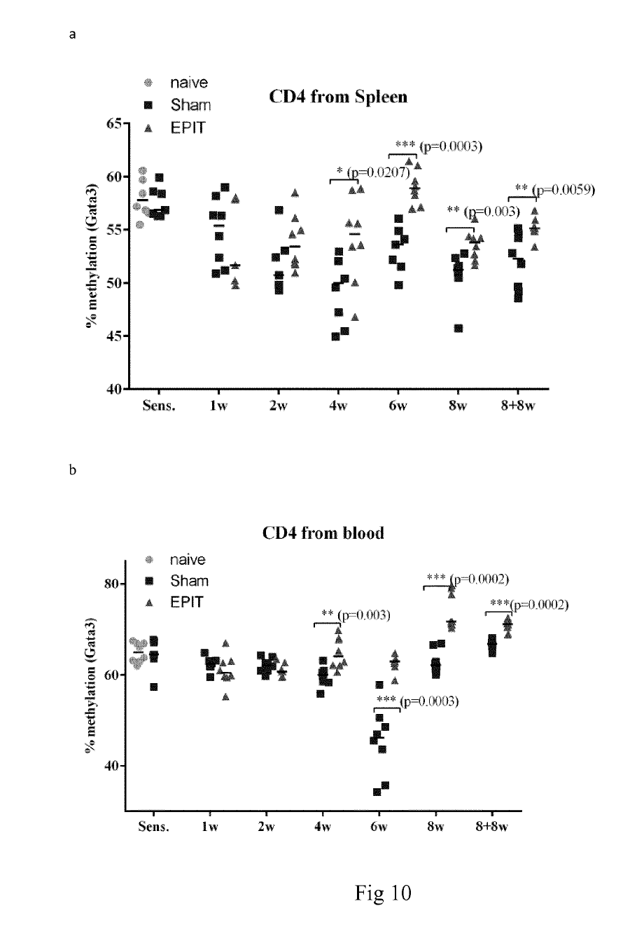

In spleen and blood CD4 cells, a significant hypermethylation of CpG island of GATA-3 occurred at the 4th week of EPIT (p<0.05 and p<0.01, respectively) and persisted after the end of EPIT (p<0.01 and p<0.001, respectively). The hypermethylation vary from 3% to 20% (FIG. 10).

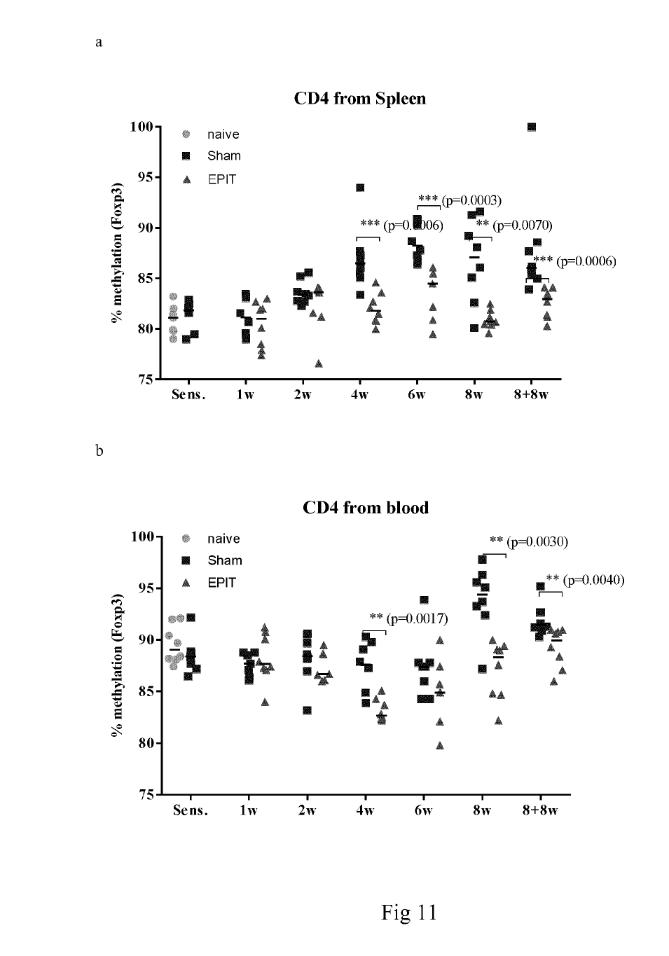

A significant hypomethylation of Foxp3 CpG islands was concomitantly obtained in spleen and blood CD4 T cells (p<0.001 and p<0.01, respectively), persisting after the end of EPIT (p<0.01) and 8 weeks after the end of EPIT® (p<0.001 and p<0.01, respectively). The hypomethylation vary from 5% to 15% (FIG. 11).

No modification was observed for these two transcription factors on CD8 and B cells and for Tbet and RORg transcription factors whatever the cells or the organs.

In conclusion, continuous EPIT acts as a strong immunomodulator, modifying the DNA expression of Th2 transcription factor by epigenetic modifications. The prolonged and continuous skin exposure of allergen through EPIT led to sustained epigenetic modifications of the DNA expression of Th2 (down regulation) and Treg (up regulation) transcription factors.

### Example 6

### Treatment of Type 1 Diabetes by Continuous EPIT

20 NOD mice (developing diabetes after 3 months of age) were sensitized to OVA and then 10 sensitized ones were EPIT-treated before developing diabetes (Group 1).

The primary endpoints are the non-development of diabetes by the measurement of glucose levels in blood. Further markers are listed in the following table. Evolution of blood glycemia was evaluated during EPIT and after discontinuation of EPIT.

| | | Immunotherapy | |
|---|---|---|---|
| | Sensiti- | (8 consecutive | |
| Groups | zation | weeks) | Markers analysed |
| 1 | OVA | EPIT OVA | Blood: Serology + glucose |
| 2 | OVA | Sham | Pancreas and liver: histology |
| 3 | NA | naive | (frozen in liquid nitrogen and formol) |
| | | | Spleen cells: 3-days in vitro |
| | | | reactivation (cytokines + Tregs + |
| | | | Bregs) |

The incidence of diabetes was significantly lower in EPIT-treated mice than in Sham or naive mice. More specifically, while 60% of naïve mice (Group 3) exhibited high glycemia and 50% of OVA-sensitized sham-treated mice (Group 2) exhibited high glycemia, upon EPIT-treatment, only 12.5% of OVA-sensitized mice exhibited high glycemia (Group 1). EPIT treatment thus prevented diabetes.

EPIT-induced Tregs specific to OVA may impact the development of diabetes in NOD mice.

1-17. (canceled)

18. A method for inducing (CTLA-4+, CD4+, CD25+) Foxp3+ Treg and/or LAP+ Treg cells in a subject, comprising applying an antigen epicutaneously to an area of the skin of the subject.

19. The method of claim 18, wherein the Treg cells are naive (CD44lo/CD62L+) Foxp3+ Tregs or effector (CD44hi/CD62L−) Foxp3+ Tregs.

20. The method of claim 18, wherein said application further induces CD304-Treg cells.

21. The method of claim 18, wherein the Treg cells further express CCR3, CCR8, CCR9, CLA, CCR6, CCR4 and/or CXCR3 molecules, and co-expression of CLA and CCR9.

22. The method of claim 18, wherein the epicutaneous application of the antigen is repeated several times or is continuous.

23. The method of claim 18, wherein the antigen is a protein, peptide, nucleic acid, lipid, particle, metal, or a combination thereof.

24. The method of claim 23, comprising (i) inducing sensitivity in a subject to an antigen and (ii) applying said antigen epicutaneously to an area of the skin of the subject under conditions sufficient to induce (CTLA-4+, CD4+, CD25)Foxp3+ Treg cells or LAP+ Tregs.

25. The method of claim 23, wherein said subject exhibits natural or induced skin sensitivity to said antigen.

26. The method of claim 18, wherein the antigen is applied in the absence of an adjuvant.

27. The method of claim 18, wherein the antigen is applied to a non-perforated and non-abraded area of the skin of the subject.

28. A method for improving a subject condition, comprising continuously applying an antigen epicutaneously to an area of the skin of the subject under conditions sufficient to induce (CTLA-4+, CD4+, CD25+)Foxp3+ Treg cells and/or LAP+ Treg cells.

29. The method of claim 30, wherein said epicutaneous application increases CpG island methylation of the GATA-3 gene and/or demethylation of the Foxp3 gene.

30. The method of claim 31, wherein said epicutaneous-mediated increase in CpG island methylation of GATA-3 gene modulates the expression of Th2 transcription factors in said subject.

31. The method of claim 30, said method reducing the onset or progression in a subject of a proliferative, autoimmune, allergic or inflammatory disease.

32. The method of claim 30, said method treating or alleviating an existing proliferative, autoimmune, allergic or inflammatory disease in the subject.

33. The method of claim 30, said method treating or alleviating type I diabetes.

34. The method of claim 30, said method treating or alleviating cancer in a subject.

35. A method for producing (CTLA-4+, CD4+, CD25+)Foxp3+ Treg cells or LAP+ Tregs, the method comprising (i) stimulating (CTLA-4+, CD4+, CD25+)Foxp3+ Tregs or LAP+ Tregs in a mammal by applying an antigen epicutaneously to an area of the skin of the mammal, (ii) collecting (CTLA-4+, CD4+, CD25+)Foxp3+ Tregs or LAP+ Tregs from said mammal, and (iii) culturing or expanding or preserving or formulating the (CTLA-4+, CD4+, CD25+)Foxp3+ Tregs or LAP+ Tregs.
